SEQUENCE LISTING (19 sequences)

SEQ ID NO: 1 - DNA, 1395 bp, Mus musculus
atgtggccgaccctgcgctacgtaggcggtgtctgcggactggcccggtactgcgtggctgggggcttcctccgcgcaagtggaccggcgtccggggtgcccgggctactgtgcgggggtggccgcagaagctccagcaccagctcttttgatatagtcatcgttggaggtggaattgtgggccttgcctctgccagaacgctcatcctgaaacatcctggactttcgattggtgtggtggaaaaggagaaagatttagctcttcaccagactggacacaacagcggtgtcatacacagtggtatttactataaaccagaatctctgaaggctaaattgtgtgtagaaggcgcagccctcatctatgagtactgcaacctcaagggaattccctacaggcaatgtggcaagctaatagtagctgttgaacaagaagaaattcccagacttcaggccttgtatgagagaggcctgcagaatggagtcgaaggcctgaggctgatccagcaggaggatataaaaaagaaggagccgtattgcaggggtctaatggctattgattgtccatacaccggcattgtgaactaccaacaggtagctttgtcatttgcccaggatttccaagaagcaggtggctctatcttgagagattttgaggtaaaaggtattgaaatagctaaagaaaactcttcaagaagtaaagatggcatgaactatccaatcgctgtcaagaacagcaagggaaaggagattcggtgtcgatatgttgtcacatgtgctggtctttactctgaccgtatttcagagttgagtggctgcaatcctgaccctcaaattgtcccattccggggagattacctggtcttaaagccagaaaagggctaccttgtaaaaggaaatatttatccggtcccagacagccggtttcctttccttggagttcactttacgcccaggttggatggcactatttggctagggcctaatgcagtgcttgcctttaaacgggaaggttacagaccctttgacttcgatgccagagatgttatggaagtaattctcaaaagcggcttcattaacctggtgttccagcatttctcttatggagttaatgaaatgtataaagcctgttttctcagtgaaacagtgaagcatcttcaaaagttcattcctgaaatcaccatcagcgatgtccttcgaggtcctgctggagtacgggcccaggccctggacagagatggaaatctggtagaggattttgtgtttgacggagggactggcgagattgcagaccgcgtccttcatgtgagaaatgcgccttcgcctgctgccacttcctccctggcaatttctagaatgatagcagaggaagcacagcaaaggtttaagttatga

SEQ ID NO: 2 - protein, 464 aa, Mus musculus
MWPTLRYVGGVCGLARYCVAGGFLRASGPASGVPGLLCGGGRRSSSTSSFDIVIVGGGIVGLASARTLILKHPGLSIGVVEKEKDLALHQTGHNSGVIHSGIYYKPESLKAKLCVEGAALIYEYCNLKGIPYRQCGKLIVAVEQEEIPRLQALYERGLQNGVEGLRLIQQEDIKKKEPYCRGLMAIDCPYTGIVNYQQVALSFAQDFQEAGGSILRDFEVKGIEIAKENSSRSKDGMNYPIAVKNSKGKEIRCRYVVTCAGLYSDRISELSGCNPDPQIVPFRGDYLVLKPEKGYLVKGNIYPVPDSRFPFLGVHFTPRLDGTIWLGPNAVLAFKREGYRPFDFDARDVMEVILKSGFINLVFQHFSYGVNEMYKACFLSETVKHLQKFIPEITISDVLRGPAGVRAQALDRDGNLVEDFVFDGGTGEIADRVLHVRNAPSPAATSSLAISRMIAEEAQQRFKL

SEQ ID NO: 3 - DNA, 1392 bp, Homo sapiens
atggtgccagcgctgcgttatttggttggtgcctgcggacgggcccgcgggcttttcgccggtggctcccctggggcgtgcgggttcgcgtctgggaggccaagaccgctgtgtggaggtagccgcagcgccagcaccagctcatttgatatagtcatcgttggtggcggaattgtggggcttgcctctgccagagcactcatcctgcgacatccatcactttctattggtgttctggaaaaggagaaagatttagctgttcaccagactggacataacagtggtgtcatacatagtggaatttattataaacctgagtctctgaaagccaaattatgtgtacaaggtgcagccctcctctatgagtactgtcagcaaaagggaatttcctacaagcagtgtggcaagcttatagtagctgttgaacaagaagaaattcccagacttcaggccctatatgagaaaggcctccagaatggtgtcccgggcctgaggctgatccagcaggaggatataaaaaagaaggagccatattgtaggggtctaatggctattgattgtccacatactggcattgtggactatcggcaggtggctttgtcatttgcccaggatttccaagaagcaggtggctctgtcttgaccaattttgaagtaaaaggtattgaaatggctaaagaaagtccttcaagaagtatagatggaatgcaatatccaattgttataaagaatacaaagggagaggaaattcgatgtcagtatgttgtgacatgtgcaggactttactcagaccgtatttcagagttgagtggctgcactcctgatcctcgaattgtaccattccggggagattacctgcttttgaagccagaaaaatgttatcttgtaaaaggaaatatttatccggtcccagatagccggtttcctttcctaggagttcacttcacaccaaggatggatggcagtatttggctagggcctaatgcagttcttgcctttaaacgagagggttacagaccctttgacttcagtgccacagatgttatggatataattatcaatagtggcttgattaaactggcatcccagaatttttcctatggagttactgaaatgtataaagcatgttttcttggtgcaacagtgaagtatcttcaaaaattcatccctgaaattactatcagtgatatacttaggggcccagctggagtaagagcccaggccctggatagagatggaaatctggtagaagattttgtatttgatgcaggagttggggatattggaaatcgcattcttcatgtgagaaatgcaccttctcctgctgctacttcttccattgcaatttctggaatgattgcagatgaagtacaacaaagatttgaattataa

SEQ ID NO: 4 - protein, 463 aa, Homo sapiens
MVPALRYLVGACGRARGLFAGGSPGACGFASGRPRPLCGGSRSASTSSFDIVIVGGGIVGLASARALILRHPSLSIGVLEKEKDLAVHQTGHNSGVIHSGIYYKPESLKAKLCVQGAALLYEYCQQKGISYKQCGKLIVAVEQEEIPRLQALYEKGLQNGVPGLRLIQQEDIKKKEPYCRGLMAIDCPHTGIVDYRQVALSFAQDFQEAGGSVLTNFEVKGIEMAKESPSRSIDGMQYPIVIKNTKGEEIRCQYVVTCAGLYSDRISELSGCTPDPRIVPFRGDYLLLKPEKCYLVKGNIYPVPDSRFPFLGVHFTPRMDGSIWLGPNAVLAFKREGYRPFDFSATDVMDIIINSGLIKLASQNFSYGVTEMYKACFLGATVKYLQKFIPEITISDILRGPAGVRAQALDRDGNLVEDFVFDAGVGDIGNRILHVRNAPSPAATSSIAISGMIADEVQQRFEL

SEQ ID NO: 5 - DNA, 6980 bp, Artificial
  pSSV9-CMV-MLC1500-L2HGDH
tggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcggtaccgcggtggcggccgcttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatgcggccgctctagctagccttgaactcactatgtaggcaagcatgaccatgaacttctgatcctccttcctcagtgtcctgggataacaggtgtgtgtcactccctacccttctaatagcaatatgtggccacatgtttgtgccccacaggttgagaccatcttgacctgaggaagaaatagctaacactcacctcctgaaggttgcctggatctcgtctttgtctttccagcactcaggagtgggggggtcagaagtgcaaagtcagcccctgctacataatgagttcaaggctcgcctgggctacatgagaccatgcctcaaaaagaaaaggaattggtatagtgacatactctggtcctcccagtacttagggacacagaggccactccaccaccatctccagcagctggcctgcctccccgagcctcgtttatttcatatcaatgagatggggacccaactgctaaggtgaccttgcacccacggggtgactggagacttgagagtggagggtttatcatttctccagtcggtcagcaagtggtcgccgccaagaaggttttgagttcaaagtagaagatgggacagggagagaccagcgagaagaccccaccctggagctgactgtccctgtgcggctgggtggggacacaaagcagagaagcagaggcagagaacaagggtgggtgacatttgagcaaggatgggggtgtgccagaggctgcccaagatgcataggtgcaaaggccctgaggttcgaggatgcctggatccggaatcaaagctcaggctcctccctcttcctcctcctcctctgccccctcctcctcctctgccccctcttcctcctctgccccctcttcttcctcctcctcttcctcctcccctcctcatctacctccttctcctcctcctccccctcctcttcctcctctgccccctcttcctcctcctcctcttcctcctcctcttcctcctcccctcctcatctacctccttctcctcctcctccccctcctcttcctcctctgccccctcttcctcctctgcccctcttcctcctcctcctcttcctcctctgccccctcctccccctcctcttcctcttcctcctcccctcctcatctacctccttctcttcctcctcttcttcctcctctttctcctcctcctccctctcctcttcctcctcctcttctttctcctcctcctcttcctccccctccccttcctgggttacttttccccattagacaatggcaggacccagagcacagagcatcgttcccaggccaggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgaatttcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcccccaattcgagctcgcccggggatcctctagcgccaccgtggagggaggggaggtggcgcaggccatgtggccgaccctgcgctacgtaggcggtgtctgcggactggcccggtactgcgtggctgggggcttcctccgcgcaagtggaccggcgtccggggtgcccgggctactgtgcgggggtggccgcagaagctccagcaccagctcttttgatatagtcatcgttggaggtggaattgtgggccttgcctctgccagaacgctcatcctgaaacatcctggactttcgattggtgtggtggaaaaggagaaagatttagctcttcaccagactggacacaacagcggtgtcatacacagtggtatttactataaaccagaatctctgaaggctaaattgtgtgtagaaggcgcagccctcatctatgagtactgcaacctcaagggaattccctacaggcaatgtggcaagctaatagtagctgttgaacaagaagaaattcccagacttcaggccttgtatgagagaggcctgcagaatggagtcgaaggcctgaggctgatccagcaggaggatataaaaaagaaggagccgtattgcaggggtctaatggctattgattgtccatacaccggcattgtgaactaccaacaggtagctttgtcatttgcccaggatttccaagaagcaggtggctctatcttgagagattttgaggtaaaaggtattgaaatagctaaagaaaactcttcaagaagtaaagatggcatgaactatccaatcgctgtcaagaacagcaagggaaaggagattcggtgtcgatatgttgtcacatgtgctggtctttactctgaccgtatttcagagttgagtggctgcaatcctgaccctcaaattgtcccattccggggagattacctggtcttaaagccagaaaagggctaccttgtaaaaggaaatatttatccggtcccagacagccggtttcctttccttggagttcactttacgcccaggttggatggcactatttggctagggcctaatgcagtgcttgcctttaaacgggaaggttacagaccctttgacttcgatgccagagatgttatggaagtaattctcaaaagcggcttcattaacctggtgttccagcatttctcttatggagttaatgaaatgtataaagcctgttttctcagtgaaacagtgaagcatcttcaaaagttcattcctgaaatcaccatcagcgatgtccttcgaggtcctgctggagtacgggcccaggccctggacagagatggaaatctggtagaggattttgtgtttgacggagggactggcgagattgcagaccgcgtccttcatgtgagaaatgcgccttcgcctgctgccacttcctccctggcaatttctagaatgatagcagaggaagcacagcaaaggtttaagttatgagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagc

SEQ ID NO: 6 - DNA, 25 bp, Artificial
  GAPDH for
atgttccagtatgactccactcacg

SEQ ID NO: 7 - DNA, 25 bp, Artificial
  GAPDH rev
gaagacaccagtagactccacgaca

SEQ ID NO: 8 - DNA, 23 bp, Artificial
  ANP for
acctgctagaccacctggaggag

SEQ ID NO: 9 - DNA, 25 bp, Artificial
  ANP rev
ccttggctgttatcttcggtaccgg

SEQ ID NO: 10 - DNA, 24 bp, Artificial
  beta-MHC for
tgcaaaggctccaggtctgagggc

SEQ ID NO: 11 - DNA, 24 bp, Artificial
  beta-MHC rev
gccaacaccaacctgtccaagttc

SEQ ID NO: 12 - DNA, 19 bp, Artificial
  RCAN1 for
gttggctggaaacaagtag

SEQ ID NO: 13 - DNA, 18 bp, Artificial
  RCAN1 rev
ggtctcttcattctctcc

SEQ ID NO: 14 - DNA, 20 bp, Artificial
  Col3 for
tggcccagctggtgacaagg

SEQ ID NO: 15 - DNA, 20 bp, Artificial
  Col3 rev
cagcagggccctttcctccc

SEQ ID NO: 16 - DNA, 20 bp, Artificial
  L2HGDH for
agggaaaggagattcggtgt

SEQ ID NO: 17 - DNA, 20 bp, Artificial
  L2HGDH rev
gggcgtaaagtgaactccaa

SEQ ID NO: 18 - DNA, 32 bp, Artificial
  NheI-L2HGDH for
tcagtcgctagcgccaccgtggagggagggga

SEQ ID NO: 19 - DNA, 29 bp, Artificial
  NheI-L2HGDH rev
tcagtcgctagccctctgccactcataac

(19) United States
(12) Patent Application Publication
(10) Pub. No.: US 2022/0111013 A1
(43) Pub. Date: Apr. 14, 2022

(54) COMPOUND DECREASING THE CONCENTRATION OF 2-HYDROXY-GLUTARATE

(71) Applicants: Universität Heidelberg, Heidelberg (DE); Christian-Albrechts-Universitat zu Kiel, Kiel (DE)

(72) Inventors: Oliver MÜLLER, Heikendorf (DE); Hugo KATUS, Heidelberg (DE); Norbert FREY, Kronshagen (DE); Lin DING, Kiel (DE); Andreas JUNGMANN, Neckargemund (DE); Anca REMES, Kiel (DE); Philipp SCHATZ, Berlin (DE); Beate KAMLAGE, Berlin (DE); Philipp TERNES, Berlin (DE)

(73) Assignees: Universität Heidelberg, Heidelberg (DE); Christian-Albrechts-Universitat zu Kiel, Kiel (DE)

(21) Appl. No.: 17/425,137

(22) PCT Filed: Jan. 23, 2020
(86) PCT No.: PCT/EP2020/051625

§ 371 Date: Jul. 22, 2021

(30) Foreign Application Priority Data
Jan. 23, 2019 (EP) 19153343.9

(51) Int. Cl. A61K 38/44; A61K 9/51; A61P 9/04

(52) U.S. Cl. CPC A61K 38/443; A61P 9/04; A61K 9/5184

(57) ABSTRACT

The present invention relates to a compound decreasing the concentration of 2-hydroxy-glutarate (2HG) in a subject for use in treating, preventing, and/or preventing progression of cardiac remodeling, in particular cardiomyopathy and/or heart failure and to viral particles, compositions, uses and methods related thereto.

The present invention relates to a compound decreasing the concentration of 2-hydroxy-glutarate (2HG) in a subject for use in treating, preventing, and/or preventing progression of cardiac remodeling, in particular cardiomyopathy and/or heart failure and to viral particles, compositions, uses and methods related thereto.

Cardiac remodeling, also referred to as ventricular remodeling, is a process changing size, shape, structure and/or function of the heart. In pathological remodeling, the remodeling process is caused by cardiomyopathies or myocardial infarction or may be caused by increased pressure in the outflow tract or volume in the ventricle, causing pressure overload or volume overload of the heart (Ponikowski et al. 2016, Goldberg 2010). Treatment of pathological cardiac remodeling includes attempts to remove the cause of remodeling, e.g. by adjusting arterial blood pressure in the patient or by correcting malfunctioning cardiac valves. Characteristically, patients suffering from cardiac remodeling are only identified once the process has become symptomatic which is upon transition of heart hypertrophy to insufficiency or even later. As a consequence, treatment typically is symptomatic without allowing for significant retardation or even reversal of the disease. This situation is aggravated by the fact that not adequately treated arterial hypertension or valvular dysfunction (stenosis) frequently lead to maladaptive remodeling of the heart muscle, in which cardiac myocytes are replaced by scar tissue. Accordingly, a significant improvement of treatment would require stopping or even reversing maladaptive remodeling as early as possible and in particular avoiding transition of hypertrophy to insufficiency.

Previously, metabolic and transcriptional changes in the myocardium of mice subjected to pressure overload by transverse aortic constriction at different time points, namely compensated hypertrophy, transition to heart failure, and end-stage heart failure, were investigated (Müller et al. 2018). Moreover, progressive alterations of key cardiac metabolic pathways and gene expression patterns associated with heart failure were identified, which in turn indicates an impaired mitochondrial function and a metabolic switch during transition to heart failure (Müller et al. 2018).

Up to now, in clinical diseases field, the accumulation of 2-hydroxyglutarate that is due to inherited mutation in IDH1/2 or L2HGDH or D2HGDH, turned out related to premature death, AML, brain tumors, and kidney cancers (Kranendijk et al. 2012, Moroni et al. 2004, Shim et al. 2014, Shelar et al. (2018)). In energy metabolism researches, the increase of 2-HG was proved to impair mitochondrial energy metabolism (Latini et al. 2005, Ban et al. (2016)) and to inhibit ATP synthase (Fu et al. 2015). Besides, it was reported that cell hypoxia could induce production of L-2-hydroxyglutarate (Intlekofer et al. 2015, Oldham et al. 2015, Bensaad et al. 2014).

Nonetheless, efficient therapies to combat maladaptive processes in the heart are highly required, in particular therapies permitting long-term treatment and/or prevention of the process. There is, thus, a need in the art for improved methods for treating cardiac remodeling. It is therefore an objective of the present invention to provide means and methods to comply with the aforementioned needs, avoiding at least in part the disadvantages of the prior art. This problem is solved by compounds, methods, and uses of the present invention. Embodiments, which might be realized in an isolated fashion or in any arbitrary combination, are listed in the dependent claims.

Accordingly, the present invention relates to a compound decreasing the concentration of L-2-hydroxy-glutarate (L2HG) in a subject for use in treating, preventing, and/or preventing progression of cardiac remodeling, in particular cardiomyopathy and/or heart failure.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may all refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a percent identity value of at least 80%, preferably at least 90%, more preferably at least 98%, most preferably at least 99%. As will be understood, the term essentially identical includes 100% identity. The aforesaid applies to the term “essentially complementary” mutatis mutandis. Unless otherwise noted, amino acid and nucleotide symbols are those of WIPO standard ST.25.

The term “2-hydroxyglutarate”, abbreviated as “2HG”, as used herein, relates to the compound known to the skilled person under this designation and also known under the chemical name 2-Hydroxypentanedioic acid, CAS No: 2889-31-8. Preferably, the 2HG is L-2-hydroxyglutarate, abbreviated as L2HG, also known under the chemical name (S)-2-Hydroxypentanedioic acid, CAS No: 13095-48-2, which is also known as L-alpha-hydroxyglutarate. The terms 2HG and L2HG, preferably, include all salts of the compounds, in particular all earth alkali and alkali salts, preferably the lithium, sodium, and potassium salts, as well as the protonated forms of 2HG and L2HG.

The term “compound decreasing the concentration of 2-hydroxyglutarate (2HG)” in a subject includes any compound causing the concentration of 2HG in a subject to decrease compared to its absence. Preferably, the compound decreasing the concentration of 2HG is an inhibitor of 2HG production. More preferably, the compound decreasing the concentration of 2HG is a compound mediating, preferably catalyzing or shifting a reaction equilibrium to increase conversion of 2HG to a different compound. Preferably, the compound decreasing the concentration of 2-hydroxyglutarate (2HG) is a compound decreasing the concentration of L-2-hydroxyglutarate (L2HG). The term “compound decreasing the concentration of L-2-hydroxy-glutarate (L2HG)” in a subject includes any compound causing the concentration of L2HG in a subject to decrease compared to its absence. Preferably, the compound decreasing the concentration of L2HG is an inhibitor of L2HG production; more preferably, the compound decreasing the concentration of 2HG is a compound mediating, preferably catalyzing or shifting a reaction equilibrium to increase conversion of 2HG to a different compound. Preferably, said inhibitor of L2HG production is a low-molecular weight compound, more preferably is malate and/or succinate, which may be administered to a subject, preferably in amounts at least twofold, more preferably at least threefold the recommended daily uptake, to shift at least one reaction equilibrium to increase removal of L2HG. In a preferred embodiment, the compound decreasing the concentration of 2HG is a malate dehydrogenase 2 (MDH2) inhibitor or a glutaminase inhibitor, preferably is an MDH2 inhibitor. Preferred MDH2 inhibitors are paullones and their derivatives; thus, in a preferred embodiment, the compound decreasing the concentration of 2HG is a 5-benzyl-paullone, wherein the benzyl-residue is preferably substituted as described by Shelar et al. (2018), and/or a paullone-9-carboxylic acid alkyl ester, preferably as described by Shelar et al. (2018). In an also preferred embodiment, the compound decreasing the concentration of 2HG is (E)-4-((4,6-dimethylpyrimidin-2-ylthio)methyl)-N′-(1-(4-methyl-3-nitrophenyl)ethylidene)benzohydrazide (AM B5965675), (E)-N-(5-acetamido-2-methoxyphenyl)-3-(3-chloro-4-isopropoxy-5-methoxyphenyl)acrylamide, LW6 (CAS NO: 934593-90-5), all three disclosed in Ban et al. (2016), or is compound 10 disclosed in Ban et al. (2016). More preferably, the inhibitor of L2HG production is an inhibitor of, preferably human, malate dehydrogenase 1 expression, of, preferably human, malate dehydrogenase 2 expression, and/or of, preferably human, lactate dehydrogenase A expression, still more preferably is an inhibitor of lactate dehydrogenase A expression, most preferably of human lactate dehydrogenase A expression. Polynucleotide inhibitors of expression of target genes are known in the art and include in particular silencing constructs, e.g. siRNAs, miRNAs, shRNAs, and the like. More preferably, the compound decreasing the concentration of L2HG is a compound degrading L2HG, preferably specifically degrading L2HG. Preferably, the compound degrading L2HG is an enzyme, more preferably, the compound decreasing the concentration of L2HG is a polypeptide having the activity of degrading L2HG. Most preferably, the compound decreasing the concentration of L2HG is L-2-hydroxyglutarate-dehydrogenase and/or a polynucleotide comprising an expressible sequence encoding an L2HGDH. The term “L-2-hydroxyglutarate-dehydrogenase”, abbreviated as “L2HGDH”, is in principle known to the skilled person to relate to an enzyme oxidizing L2HG, preferably of oxidizing L2HG to 2-Ketoglutarate (2-Oxo-glutatarate). Thus, preferably, the L-2-hydroxyglutarate-dehydrogenase is an enzyme of E.C. 1.1.99.2. Preferably, L2HG concentration is decreased in blood and/or intracellularly in cells of the circulatory system, preferably in heart muscle cells (cardiomyocytes). More preferably, the L2HGDH has the sequence of Genbank Acc No. NP_663418.1 or NP_079160.1.

The term “polynucleotide”, as used herein, refers to a linear or circular nucleic acid molecule. The polynucleotide of the invention comprises an expressible sequence encoding a 2HGDH, preferably an L2HGDH, i.e. an enzyme having 2HG degrading activity as specified herein above, and/or a polynucleotide inhibitor of 2HG, preferably 12HG, production, more preferably comprises an expressible sequence encoding an 2HGDH, even more preferably an L2HGDH; thus, the polynucleotide has the biological activity of causing a decrease of 2HG concentration in a living cell when introduced therein. The aforesaid activity of the polynucleotide can be established by methods known in the art and as described herein in the examples. More preferably, the polynucleotide comprises a nucleic acid sequence corresponding to at least one of the sequences of SEQ ID NOs:1 and 3, preferably encoding polypeptides having amino acid sequences corresponding to the sequences of SEQ ID NOs:2 and 4, respectively. More preferably, the polynucleotide comprises a coding sequence derivable from Genbank Acc No: NM_145443.2 and/or NM_024884.3, still more preferably, the polynucleotide comprises, preferably consists of, an expression construct for a 2HGDH comprised in an AAV vector; thus, most preferably, the polynucleotide comprises, preferably consists of, the nucleic acid sequence of SEQ ID NO:5. The term “polynucleotide” encompasses single as well as partially or completely double-stranded polynucleotides. Preferably, the polynucleotide is RNA or is DNA, including cDNA. Preferably, the polynucleotide is a DNA. More preferably, the DNA polynucleotide further comprises at least one promoter causing expression of an RNA encoding a polypeptide having an activity as specified herein above. The skilled person is able to select an appropriate promoter for such purpose; preferably, the promoter is an inducible promoter, more preferably, the promoter is a constitutive promoter. Also preferably, the promoter is a cell-type specific promoter, in particular a cardiomyocyte-specific promoter. Also preferably, the polynucleotide is an RNA. Preferably, said RNA is an mRNA, preferably expressed or expressible from the DNA polynucleotide comprising a promoter as specified herein above. Moreover, comprised are also chemically modified polynucleotides including naturally occurring modified polynucleotides such as glycosylated or methylated polynucleotides or artificially modified derivatives such as biotinylated polynucleotides or polynucleotides comprising phosphorothioates. The polynucleotide of the present invention shall be provided, preferably, either as an isolated polynucleotide (i.e. isolated from its natural context) or in genetically modified form. Preferably, the polynucleotide has a length of at most 1 Mb, more preferably at most 100 kb, even more preferably at most 10 kb, still more preferably at most 5 kb, most preferably at most 5 kb. Preferably, the polynucleotide is a non-naturally occurring polynucleotide; thus, preferably, the nucleotide is an artificial polynucleotide. Also preferably, the polynucleotide is a chimeric polynucleotide; more preferably, the polynucleotide comprises at least one nucleic acid sequence heterologous to the remaining nucleic acid sequences it comprises. As used herein, the term polynucleotide, preferably, includes variants of the specifically indicated polynucleotides. More preferably, the term polynucleotide relates to polynucleotides essentially identical to the specific polynucleotides indicated. Most preferably, the term polynucleotide relates to the specific polynucleotides indicated. The term “polynucleotide variant”, as used herein, relates to a variant of a polynucleotide related to comprising a nucleic acid sequence characterized in that the sequence can be derived from the aforementioned specific nucleic acid sequence by at least one nucleotide substitution, addition and/or deletion, wherein the polynucleotide variant shall have the biological activity or activities as specified for the specific polynucleotide. Thus, it is to be understood that a polynucleotide variant as referred to in accordance with the present invention shall have a nucleic acid sequence which differs due to at least one nucleotide substitution, deletion and/or addition. Preferably, said polynucleotide variant comprises an ortholog, a paralog or another homolog of the specific polynucleotide or of a functional subsequence thereof. Also preferably, said polynucleotide variant comprises a naturally occurring allele of the specific polynucleotide or of a functional subsequence thereof. Polynucleotide variants also encompass polynucleotides comprising a nucleic acid sequence which is capable of hybridizing to the aforementioned specific polynucleotides or functional subsequences thereof, preferably, under stringent hybridization conditions. These stringent conditions are known to the skilled worker and can be found in standard textbooks. A preferred example for stringent hybridization conditions are hybridization conditions in 6× sodium chloride/sodium citrate (═SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide; accordingly, other conditions more suitable for low-G+C DNA, which are in principle known to the skilled person, may be found to be more appropriate by the skilled person. The skilled worker knows how to determine the hybridization conditions required by referring to standard textbooks. Alternatively, polynucleotide variants are obtainable by PCR-based techniques such as mixed oligonucleotide primer-based amplification of DNA, e.g. using degenerated primers. As a template, DNA or cDNA from bacteria, fungi, plants or, preferably, from animals may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences or functional subsequences thereof. The percent identity values are, preferably, calculated over the entire nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981))), are preferably used. Preferably, said programs are used with their standard parameters. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. A polynucleotide comprising a fragment of any of the specifically indicated nucleic acid sequences, said polynucleotide retaining the indicated activity or activities, is also encompassed as a variant polynucleotide of the present invention. A fragment as meant herein, preferably, comprises at least 300, preferably at least 1000, more preferably at least 2000 consecutive nucleotides of any one of the specific nucleic acid sequences and still has the indicated activity. The polynucleotides of the present invention either consist of, essentially consist of, or comprise the aforementioned nucleic acid sequences. Thus, they may contain further nucleic acid sequences as well. Specifically, the polynucleotide of the present invention may encode e.g. further polypeptides, including fusion polypeptides and selectable markers. Such fusion polypeptides may comprise as additional part polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and are described elsewhere herein. Selectable markers are known in the art and include in particular antibiotic resistance genes, selectable metabolic markers, e.g. an auxotrophy marker, and the like. More preferably, the polynucleotide comprises at least one further transcription factor binding site sequence and a corresponding reverse complement of said at least one further transcription factor binding site sequence. Preferably, the polynucleotide further comprises at least one sequence mediating packaging of said polynucleotide into a viral particle; thus, preferably, the polynucleotide can preferably be provided packaged into a viral particle. Preferably, said viral particle is a replication-incompetent viral particle, e.g. a virus-like particle (VLP). Packaging sequences for relevant viruses are well-known in the art. Preferably, the virus is an adeno-associated virus (AAV), an adenovirus, a retrovirus, preferably a HIV-derivative, or a herpesvirus. Preferably, the virus is an adeno-associated virus; thus, preferably, the viral particle is an adeno-associated virus-like particle (AA-VLP), preferably AAV9. In a preferred embodiment, the viral particle is an AAV6 virus of an AAV6 VLP. AA-VLPs are known in the art, including derivatives having a modified cellular tropism compared to wildtype AAV.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. Furthermore, the term, preferably, includes conservative treatment, i.e. treatment preventing or impeding aggravation of a disease or disorder or a symptom thereof. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well-known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20%, at least 50%, at least 60%, at least 70%, at least 80%, or at least 90% of the subjects of a given cohort or population. As will be understood by the skilled person, effectiveness of treatment of e.g. cardiac remodeling is dependent on a variety of factors including, e.g. severity of existing cardiac remodeling, accompanying diseases, and further risk factors. Preferably, treating comprises decreasing L2HG concentration in affected cells. Preferably, treating a disease or condition with a compound recited in this specification consists of a single administration of said compound within a long period of time, preferably six months, more preferably one year, most preferably two years, i.e., preferably, is a long-term treatment. More preferably, treating a disease or condition with a compound recited in this specification consists of a single administration of said compound, i.e., preferably, is a one-time treatment. The treatment, preferably, includes additional therapeutic measures, e.g. administration of calcineurin inhibitors. Preferably, in case the disease is cardiac remodeling, said treatment further comprises administration of at least one drug selected from the group consisting of ACE Inhibitors (ACEI), Beta Blockers, AT1-Inhibitors, Aldosteron Antagonists, Renin Antagonists, Diuretics, Ca-Sensitizers, Digitalis Glycosides, polypeptides of the protein S100 family, and natriuretic peptides such as BNP (Nesiritide (human recombinant Brain Natriuretic Peptide—BNP)) or ANP.

The term “preventing” refers to retaining health with respect to the diseases or disorders referred to herein for a certain period of time in a subject. It will be understood that said period of time may be dependent on the amount of drug compound which has been administered and on individual factors of the subject discussed elsewhere in this specification. It is to be understood that prevention may not be effective in all subjects treated with the compound. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well-known statistic evaluation tools discussed elsewhere in this specification. Preferably, preventing a disease or condition with a compound recited in this specification consists of a single administration of said compound, in particular to a subject at risk for developing said disease or condition, within a long period of time, preferably six months, more preferably one year, most preferably two years, i.e., preferably, is a long-term preventive treatment. More preferably, preventing a disease or condition with a compound recited in this specification consists of a single administration of said compound, i.e., preferably, is a one-time preventive treatment.

The term cardiac remodeling, is known to the skilled person to relate to changes in the heart's size and shape that occur in response to cardiac disease or cardiac damage; the term in particular includes pathological cardiac remodeling, in particular cardiomyopathy and/or heart failure. Thus, preferably, the tissue in which 2HG concentration shall be decreased is a tissue of the cardiovascular system, preferably of the heart; more preferably, 2HG concentration shall be decreased in cardiac muscle cells (cardiomyocytes). Thus, preferably, the disease referred to herein is cardiac hypertrophy and/or cardiomyopathy and/or heart failure. Conditions, symptoms thereof are, preferably, (i) arterial hypertension; (ii) congenital, age-related degenerative, or infection-related semilunar valve stenosis, in particular aortic valve stenosis; (iii) cardiomyopathy, in particular dilated cardiomyopathy, hypertrophic cardiomyopathy, arrhythmogenic right ventricular cardiomyopathy, left ventricular noncompaction, or restrictive cardiomyopathy; (iv) coronary heart disease; or (v) myocarditis. Preferably, coronary heart disease is associated with increased risk of myocardial infarction, more preferably myocardial infarction with subsequent cardiac remodeling (ischemic heart disease).

As used herein, the term “subject” relates to a vertebrate. Preferably, the subject is a mammal, more preferably, a mouse, rat, cat, dog, hamster, guinea pig, sheep, goat, pig, cattle, or horse. Still more preferably, the subject is a primate. Most preferably, the subject is a human. Preferably, the subject is afflicted with or having an increased risk of becoming afflicted with cardiac remodeling.

Advantageously, it was found in the work underlying the present invention that increased amounts of L2HG contribute to cardiac remodeling in a heart failure model and, surprisingly, removal of L2HG by enzymatic degradation helps to alleviate its symptoms and to prevent cardiac remodeling.

The definitions made above apply mutatis mutandis to the following. Additional definitions and explanations made further below also apply for all embodiments described in this specification mutatis mutandis.

The present invention further relates to a polynucleotide according to the present invention for use in treating and/or preventing disease.

The present invention also relates to a viral particle comprising a polynucleotide comprising an expressible sequence encoding an L2HGDH. Preferably, said viral particle is for use in treating, preventing, and/or preventing progression disease, in particular cardiac remodeling, preferably cardiomyopathy and/or heart failure.

The term “viral particle” is understood by the skilled person and includes all types of particles derived from any virus comprising at least one virus-encoded polypeptide and mediating entry of a polynucleotide or polynucleotides associated therewith into a suitable host cell. Preferably, the viral particle comprises at least a capsid comprising the aforesaid polynucleotide or polynucleotides. Preferably, the viral particle is a viral particle derived from a virus neither replicating lytically nor establishing latent infection in a suitable host cell; i.e. preferably, the viral particle is a virus-like particle as known from the art. Preferably, the viral particle is an adeno-associated virus particle. Thus, preferably, the viral particle is an adeno-associated VLP (AAV-VLP), preferably is an AAV9-VLP; in a preferred embodiment, the viral particle is an AAV6-VLP.

The present invention also relates to a composition comprising viral particles of the present invention.

The term “composition”, as used herein, relates to a mixture of compounds comprising at least viral particles as specified herein and at least one carrier. The carrier(s) must be acceptable in the sense of being compatible with the other ingredients of the composition and being not deleterious to a potential recipient thereof. The carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Suitable carriers comprise those mentioned above and others well known in the art. The carrier(s) is/are selected so as not to affect the biological activity of the composition.

Examples of such diluents are distilled water, physiological saline, Ringer's solutions, dextrose solution, and Hank's solution. Preferably, in the composition, at least 50%, preferably at least 75%, more preferably at least 90%, even more preferably at least 95%, most preferably essentially all viral particles are viral particles comprising a polynucleotide as specified elsewhere herein.

Preferably, the composition is a pharmaceutical composition; thus, preferably, the carrier is a pharmaceutically acceptable carrier. In addition, the pharmaceutical composition or formulation may also include other carriers, adjuvants, or nontoxic, nontherapeutic, non-immunogenic stabilizers and the like. The polynucleotide of the present invention can be formulated as a pharmaceutically acceptable salt. Acceptable salts comprise acetate, methylester, HCl, sulfate, chloride and the like. The pharmaceutical compositions are, preferably, administered topically or systemically. Suitable routes of administration conventionally used for drug administration are oral, intravenous, or parenteral administration as well as inhalation. However, depending on the nature and mode of action of a compound, the pharmaceutical compositions may be administered by other routes as well, in particular as specified elsewhere herein. Moreover, the polynucleotide can be administered in combination with other drugs either in a common pharmaceutical composition or as separated pharmaceutical compositions wherein said separated pharmaceutical compositions may be provided in form of a kit of parts. The binding polypeptide is, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

A therapeutically effective dose refers to an amount of the polynucleotide to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and other clinical factors; preferably in accordance with any one of the above described methods. As is well known in the medical arts, dosages for any one patient depend upon many factors, which may include the patient's size, body surface area, age, the particular compound to be administered, sex, time and route of administration, general health, and other drugs being administered concurrently. Progress can be monitored by periodic assessment. A typical dose can be, for example, in the range of 1 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 1 mg units per kilogram of body weight per minute, respectively. Progress can be monitored by periodic assessment. However, depending on the subject and the mode of administration, the quantity of substance administration may vary over a wide range to provide from about 0.01 mg per kg body mass to about 10 mg per kg body mass. The pharmaceutical compositions and formulations referred to herein are administered at least once in order to treat or ameliorate or prevent a disease or condition recited in this specification. However, the said pharmaceutical compositions may be administered more than one time, for example from one to four times daily up to a non-limited number of days. Preferably, treating or preventing a disease or condition with a compound recited in this specification consists of a single administration of said compound.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the prescribers or user's instructions in order to anticipate dose adjustments depending on the considered recipient.

The present invention further relates to a use of a viral particle according the present invention and/or a composition according to the present invention for decreasing L2HG-concentration in a cell.

The present invention also relates to a method for decreasing the concentration of 2-hydroxy-glutarate (2HG), preferably of L2HG, in a subject, comprising contacting said subject with a compound as specified herein above; and thereby decreasing the concentration of L2HG in a subject.

The method, preferably, is an in vitro method; it may, however, also be performed in vivo, in which case said method preferably is a method for treating, preventing, and/or preventing progression of cardiac remodeling. Moreover, the method may comprise steps in addition to those explicitly mentioned above. For example, further steps may relate, e.g., to providing a viral particle according to the present invention for step a), or additional treatment of the disease in step b). Moreover, one or more of said steps may be performed by automated equipment.

Preferably, the method comprises topical and/or systemic application of the viral particles. Preferably, the topical application is epicutaneous. Systemic application, preferably, comprises transcutaneous, intraarterial, or intravenous application. As will be understood by the skilled person, the preferred mode of administration will depend on the type of disease and, thus, the target tissue; e.g. cardiac remodeling, the preferred mode of administration will be intravenous or intracardial. Preferably, intraarterial or intravenous application is catheter-assisted. It is however, also envisaged that the method comprises systemic administration of the viral particles, e.g. by intravenous infusion.

In view of the above, the following embodiments are particularly envisaged:

Embodiment 1: A compound decreasing the concentration of 2-hydroxy-glutarate (2HG) in a subject for use in treating, preventing, and/or preventing progression of cardiac remodeling, in particular cardiomyopathy and/or heart failure.

Embodiment 2: The compound of embodiment 1, wherein said 2HG is L-2-hydroxy-glutarate (L2HG).

Embodiment 3: The compound of embodiment 1 or 2, wherein said concentration is blood concentration and/or intracellular concentration in cells of the circulatory system.

Embodiment 4: The compound of any one of embodiments 1 to 3, wherein said concentration is concentration in cells of the heart, preferably in cardiomyocytes.

Embodiment 5: The compound of any one of embodiments 1 to 4, wherein said compound is an enzyme catalyzing degradation of 2HG, preferably is an 2HG-Dehydrogenase (2HGDH); or is a compound inhibiting 2HG production, preferably a 5-benzyl-paullone, a paullone-9-carboxylic acid alkyl ester, (E)-4-((4,6-dimethylpyrimidin-2-ylthio)methyl)-N′-(1-(4-methyl-3-nitrophenyl)ethylidene)benzohydrazide (AM B5965675), (E)-N-(5-acetamido-2-methoxyphenyl)-3-(3-chloro-4-isopropoxy-5-methoxyphenyl)acrylamide, or LW6 (CAS NO: 934593-90-5).

Embodiment 6: The compound of embodiment 5, wherein said compound is an enzyme catalyzing degradation of L2HG, preferably is an L2HG-Dehydrogenase (L2HGDH).

Embodiment 7: The compound of embodiment 5 or 6, wherein said compound is an L2HGDH enzyme EC 1.1.99.2.

Embodiment 8: The compound of any one of embodiments 1 to 7, wherein said compound is a polynucleotide comprising an expressible sequence encoding a 2HGDH, preferably an L2HGDH.

Embodiment 9: The compound of any one of embodiments 1 to 8, wherein said compound is a polynucleotide comprising an expressible sequence encoding a 2HGDH, preferably an L2HGDH, comprised in a viral particle, preferable an adeno-associated virus (AAV) particle, more preferably in an AAV9 particle, or in an AAV6 particle.

Embodiment 10: The compound of any one of embodiments 1 to 9, wherein said polynucleotide comprises
(i) the nucleic acid sequence of SEQ ID NO:1 and/or 3 or a nucleic acid sequence at least 70% identical to at least one of SEQ ID NO:1 and/or 3; and/or
(ii) a nucleic acid sequence encoding a polypeptide comprising the amino acid sequence of SEQ ID NO: 2 and/or 4; or encoding a polypeptide comprising an amino acid sequence at least 70% identical to at least one of SEQ ID NO:2 and/or 4.

Embodiment 11: The compound of any one of embodiments 1 to 10, wherein said polynucleotide comprises the nucleic acid sequence of SEQ ID NO:5 or a nucleic acid sequence at least 70% identical thereto; preferably comprises the nucleic acid sequence of SEQ ID NO:5, more preferably consists of the nucleic acid sequence of SEQ ID NO:5.

Embodiment 12: The compound of any one of embodiments 1 to 11, wherein said cardiac remodeling is caused by (i) arterial hypertension; (ii) congenital, age-related degenerative, or infection-related semilunar valve stenosis, in particular aortic valve stenosis; (iii) cardiomyopathy, in particular dilated cardiomyopathy, hypertrophic cardiomyopathy, arrhythmogenic right ventricular cardiomyopathy, left ventricular noncompaction, or restrictive cardiomyopathy; (iv) coronary heart disease; or (v) myocarditis.

Embodiment 13: The compound of any one of embodiments 1 to 12, wherein said subject is a mammal, preferably a human.

Embodiment 14: A viral particle comprising a polynucleotide comprising an expressible sequence encoding a 2HGDH, preferably an L2HGDH.

Embodiment 15: The viral particle of embodiment 14, wherein said viral particle is a virus-like particle.

Embodiment 16: The viral particle of embodiment 14 or 15, wherein said viral particle is an adeno-associated virus (AAV) particle, more preferably is an AAV9 particle or an AAV6 particle.

Embodiment 17: A composition comprising viral particles according to any one of embodiments 14 to 16.

Embodiment 18: The composition of embodiment 17, wherein said composition is a pharmaceutical composition.

Embodiment 19: A viral particle according to any one of embodiments 14 to 16 and/or a composition according to embodiment 17 or 18 for use in treating, preventing, and/or preventing progression of disease.

Embodiment 20: A viral particle according to any one of embodiments 14 to 16 and/or a composition according to embodiment 17 or 18 for use in treating, preventing, and/or preventing progression of cardiac remodeling, in particular cardiomyopathy and/or heart failure.

Embodiment 21: The viral particle for use according to embodiment 20 or 21 and/or a composition for use according to embodiment 20 or 21, wherein said viral article is a viral particle as specified in any one of embodiments 1 to 14.

Embodiment 22: Use of viral particle according to any one of embodiments 14 to 16 and/or a composition according to embodiment 17 or 18 for decreasing 2HG-concentration, preferably L2HG-concentration, in a cell.

Embodiment 23: The use of embodiment 22, wherein said use is an in vitro use.

Embodiment 24: A method for decreasing the concentration of 2HG, preferably L2HG, in a subject, comprising
(i) contacting said subject with a compound as specified in any one of embodiments 1 to 14; and
(ii) thereby decreasing the concentration of 2HG, preferably L2HG, in a subject.

Embodiment 25: The method of embodiment 24, wherein said decreasing the concentration of 2HG in a subject is treating, preventing, and/or preventing progression cardiac remodeling, in particular cardiomyopathy and/or heart failure.

Embodiment 26: A kit comprising a (i) polynucleotide comprising an expressible sequence encoding a 2HGDH, preferably an L2HGDH, and at least one viral packaging signal, and (ii) a corresponding packaging helper polynucleotide and/or a corresponding packaging cell line.

All references cited in this specification are herewith incorporated by reference with respect to their entire disclosure content and the disclosure content specifically mentioned in this specification.

### FIGURE LEGENDS

FIG. 1. (A) Gene expression analysis of L2HDGH in Dyrk1a over-expression transgenic mice model, Ang-II infusion mice model and TAC operated cardiac hypertrophy mice model. (B) Representative Western blot image of L2HGDH expression on protein level in heart homogenates of left ventricular 6 weeks subjected to sham/TAC and corresponding statistical quantification (C) (n=5-7 mice/group. *p<0.05; **p<0.01; ns=not significant).

FIG. 2. Contractile function alterations in response to TAC/Sham. (A) Time-course monitoring of systolic function including ejection fraction and fractional shortening by echocardiography. Left ventricular end-diastolic diameter and left ventricular mass were determined as a measure of hypertrophic response in the different treatment groups. *p<0.05, **p<0.01, ***p<0.001: Sham versus AAV9-LUC+TAC. #<0.05, ##p<0.01, ###p<0.001: Sham versus AAV9-L2HGDH+TAC. § <0.05, §§ p<0.01, § § § p<0.001: AAV9-LUC+TAC versus AAV9-L2HGDH+TAC. (B) Representative short axis M-mode echocardiographs from 4 weeks post-TAC/Sham mice of the three groups.

FIG. 3. Histological analysis of hypertrophy and fibrosis, fibrosis markers expression. Statistical analysis was performed with one-way ANOVA multiple comparison including a Student-Newman-Keuls' post hoc analysis. (A) Gross morphology of heart sections from the mentioned treatment groups and corresponding representative images of HE stainings. (B) Statistical quantification of relative cardiomyocyte area in the depicted groups. (C) Masson's Trichrome staining showing extracellular matrix deposion in perivascular and interstitial areas. (D) Statistical quantification of the percentage of fibrosis (blue area) of at least 10 randomly selected areas in sections of myocardium in the indicated treatment groups. Scale bar represents 20 μm. (E) Gene expression analysis of fibrosis markers collagen 3a1 and TGF-β in cardiac tissue. mRNA levels were normalized to RPL32 as a housekeeping gene (n=14/14/12 mice in sham/AAV9-LUC/AAV9-L2HGDH groups, *** p<0.001; &p<0.05 vs. Sham control; #p<0.05 vs. AAV9-LUC+TAC).

FIG. 4. Measurement of Mitochondrial Superoxide Levels (MSLs) with MitoSOX in NRVCM. (A) Representative immunofluorescence images of cells belonging to the mentioned groups. Red represents MitoSOX fluorescence. DAPI (blue) was used to mark cell nuclei. Scale bars represent 20 μm. (B) Statistical quantification of MFIs (p<0.001 for the virus treatment and Interaction). 6-8 images/slide, n=5-6 slide/group. (&p<0.001 vs. Ad-LacZ+H2O group; #p<0.001 vs. Ad-LacZ+ET-1 group).

FIG. 5. Cell surface area measurement in NRVCM. (A) Representative immunofluorescence images of alpha-actinin staining (red) in cells receiving the four mentioned treatments. DAPI (blue) was used as a nuclear marker. Scale bar represents 50 μm. (B) Statistical quantification of cell surface area (p<0.001 for the virus infection treatment, hypertrophic stimulation treatment and interaction). (10 images/slide, n=3 slides/group. &p<0.001 vs. Ad-LacZ+H2O group; #p<0.001 vs. Ad-LacZ+ET-1 group).

FIG. 6. Pro-hypertrophic effects of 2-HG. (A) Representative immunofluorescence images alpha-actinin staining (red) and DAPI (blue). Scale bar represents 50 μm. (B) Statistical quantification of ANP and BNP mRNA levels, using RPL32 as a housekeeping gene. (10 images/slide, n=3 slides/group, **p<0.01. (C) Statistical quantification of relative level of 5-hmC following 2-HG application.

FIG. 7. L2HGDH is overexpressed in the mitochondria following AAV6 transduction. (A) Representative image of Western blot experiments using total NRVCMs protein lysates. GAPDH was used as an internal control, proving equal protein loading. (B) Statistical quantification of L2HGDH protein level in NRVCMs. Values were normalized to control non-transduced cells. (C) Illustrative confocal images of L2HGDH immunocytochemistry and Mitotracker, specifically labelling mitochondria. Scale bar represents 100 μm. (20 images/experimental group, n=4, ***p<0.001).

FIG. 8. 2HG acts as a pro-hypertrophic marker, while L2HGDH overexpression rescues the observed phenotype. (A) Representative images of α-actinin immunocytochemistry (red) performed using NRVCMs subjected to the mentioned treatments. Scale bar represents 25 μm. (B) Statistical quantification of cell area as a marker of pro-hypertrophic effect of L-2HG. (C, D) Gene expression analysis of ANP and BNP as hypertrophic markers, analysed by RT qPCR. RPL32 was used as a housekeeping gene. (40 images/experimental group, n=4, * p<0.05 **p<0.01).

FIG. 9. L2HGDH ameliorates 2HG-induced mitochondria dysfunction in NRVCMs. (A) Representative images showing live cell monitoring of mitochondrial membrane potential (red) at the mentioned time points and (B) statistical quantification of red fluorescence intensity. (C) Illustrative confocal images of NRVCMs stained with JC-1 as a marker for mitochondria function. Red fluorescence depicts polarized mitochondria, while 2HG induced depolarization causes a shift to detectable green fluorescence. (D) statistical quantification of mean green/red fluorescence intensities in the depicted treatment groups.

FIG. 10. 2HG induces fibroblast proliferation and collagen deposition in vitro. (A) Representative Western blot image showing collagen3a1 expression in total lysate isolated from primary cardiac fibrolasts. beta-actin was used as a loading control, proving equal protein loading. (B) Statistical quantification of collagen3a1 protein level. Non-treated fibroblasts were used as control. (C) Illustrative images showing Ki67 expression in fibroblasts by immunocytochemistry. DAPI was used as a nuclear marker. Scale bar represents 50 μm. (D) Quantification of the percentage of Ki67-positive nuclei in immunocytochemistry images. (20 images/experimental group, n=4, **p<0.01)

The following Examples shall merely illustrate the invention. They shall not be construed, whatsoever, to limit the scope of the invention.

### EXAMPLE 1: MATERIALS AND METHODS

### Data Integration to a Multi-Omics Data Set and Filter Approaches for Potential Drug Target Identification

To identify relations between genes and metabolites, ANOVA results of metabolomics and transcriptomics data sets were integrated to a multi-omics data set. Metabolites were mapped to human proteins based on protein associations (comprising mainly enzymes, transporters, and receptors) downloaded from the Human Metabolome Database (HMDB, www.hmdb.ca). Subsequently, human proteins were mapped to mouse genes using HomoloGene (www.ncbi.nlm.nih.gov/homologene) group identifiers. This relationship of functionally related metabolite-gene pairs was filtered for interesting common regulations by application of two filters. Filter 1_up: Significant upregulation of mRNA in all three time points relative to sham control and significant change of related metabolite in all three time points relative to sham control. The direction of metabolite change was either up- or downregulated, however, with consistent direction in all three time points. Filter 1_down: Significant downregulation of mRNA in all three time points relative to sham control and significant change of related metabolite in all three time points relative to sham control. The direction of metabolite change was either up- or downregulated, however, with consistent direction in all three time points.

Upregulation means a significant (p<0.05) fold change relative to sham control >1.1 and downregulation a significant (p<0.05) change relative to sham control <0.9.

### Vector Cloning

The murine L2HGDH cDNA was amplified using the following primers with a NheI restriction site of each (tcagtcgctagcgccaccGTGGAGGGAGGGGA (SEQ ID NO:18)) and tcagtcgctagcCCTCTGCCACTCATAAC SEQ ID NO:19). 100 ng of murine cDNA was used to amplify the L2HGDH sequence following restriction with NheI, then inserted into a single stranded AAV genome plasmid (pSSV9) that contains the cardiac specific and CMV-enhanced MLC1500 promoter (Müller et al. 2007). Luciferase was used as control gene.

### Vector Production and Quantification

Vector production was carried out as published earlier using the two plasmid system (Werfel et al. 2014). In brief, the AAV genome plasmid pSSV9-CMV-MLC1500-mL2HGDH was co-transfected with pDP9rs, that encodes the adenoviral helper genes for the serotype 9. After 72 hours, the vectors were harvested and purified with a discontinuous iodixanol gradient via ultracentrifugation. Quantification was done by qPCR.

RNA Isolation, cDNA Synthesis and Relative Quantification qRT-PCR

RNA was extracted and purified from frozen mouse heart using Trizol reagent (QIAzol lysis reagent, Qiagen) following manufacturer's instructions, followed by DnaseI digestion. Nucleic acid yields were analyzed photometrically (NanoDrop ND-1000, Spectrophotometer). 1.5 μg RNA was reverse transcribed into cDNA using Superscript III Kit (Invitrogen) and oligo(dT) Primers. cDNA synthesis was followed by RNA digestion using RNase H. qRT-PCR was executed using iTaq Universal SYBR Green Supermix (Thermo Fisher Scientific) and CFX96 Touch Real-Time PCR detection system (Bio Rad) as previously described (Doehner 2014). The following primers used for cDNA relative quantification are listed below.

| Gene | Sequence | SEQ ID NO: |
|---|---|---|
| GAPDH | forward: | 6 |
| | 5′-ATGTTCCAGTATGACTCCACTCACG-3′ | |
| | reverse: | 7 |
| | 5′-GAAGACACCAGTAGACTCCACGACA-3′ | |
| ANP | forward: | 8 |
| | 5′-ACCTGCTAGACCACCTGGAGGAG-3′ | |
| | reverse: | 9 |
| | 5′-CCTTGGCTGTTATCTTCGGTACCGG-3′ | |
| β-MHC | forward: | 10 |
| | 5′-TGCAAAGGCTCCAGGTCTGAGGGC-3′ | |
| | reverse: | 11 |
| | 5′-GCCAACACCAACCTGTCCAAGTTC-3′ | |
| RCAN1 | forward: 5′-GTTGGCTGGAAACAAGTAG-3′ | 12 |
| | reverse: 5′-GGTCTCTTCATTCTCTCC-3′ | 13 |
| Col3 | forward: 5′-TGGCCCAGCTGGTGACAAGG-3′ | 14 |
| | reverse: 5′-CAGCAGGGCCCTTTCCTCCC-3′ | 15 |
| L2HGDH | forward: 5′-AGGGAAAGGAGATTCGGTGT-3′ | 16 |
| | reverse: 5′-GGGCGTAAAGTGAACTCCAA-3′ | 17 |

Two technical replicates were considered for each reaction. GAPDH served as a housekeeping gene, and values were normalized to the sham group.

### Protein Isolation and Western Blot Analysis

Protein was extracted from cardiac tissue using RIPA buffer containing a mixture of protease inhibitors, followed by Western blot analysis as previously described with slight alterations (Heckmann et al. 2016). A rabbit anti-L2HGDH antibody was used (LS-C165661-400, LS Bio, Washington, USA) and incubated at a dilution of 1:1000. Images were analysed using ImageLab (Bio-Rad, California, USA).

### Histological Analysis

Heart tissue was fixed in 4% PFA overnight at 4° C. and embedded in paraffin. For cell surface area measurement, the sections were stained Hematoxylin and Eosin staining as published before (Fischer et al. 2008). For visualization of extracellular matrix deposition, sections were subjected to Masson's Trichrome staining as previously described (Bickelhaupt et al. 2017). Images were taken in random areas of the left ventricle using a bright field microscope (Leica DM500, Leica Microsystems, Mannheim, Germany). Cell surface area was analysed using Image J (NIH, Bethesda, Md., USA) and the average value of each group was calculated. Interstitial fibrosis and perivascular fibrosis were quantified using Image Dx (Reveal Biosciences, San Diego, Calif.).

### Study Protocol and Animal Handling

8 weeks old male wild-type mice (C57BL/6NCrl—Charles River) were randomly assigned to treatment (AAV9-L2HGDH, n=16) or vector control group (AAV9-LUC, n=19) with a dose of 10¹² vector genomes/mouse (injected into the tail vein) for both groups. Another 15 mice served as sham control without any injection. Two weeks after injection, mice in the AAV9-L2HGDH and AAV9-LUC groups were subjected to TAC surgery (deAlmeida et al. 2010). A 27-gauge needle was used for inducing the stenosis. Successful ligation was confirmed by echocardiography measurements of the innominate artery/left common carotid artery flow velocity ratio during 48-72 hours after TAC surgery. Prior to TAC procedure as well as every two weeks after TAC, left ventricular function was monitored using echocardiography (Vevo2100 System), under anaesthesia. 6 weeks after TAC, mice were sacrificed and tissue samples were harvested. All clinical parameters were measured and analysed by blinded investigators. Mice were housed in pathogen-free conditions with controlled temperature and humidity and day/night rhythm of 12:12 hours. A complete diet of Rod 16-A (LASvendi, Soest, Germany) and water were served ad libitum. All animal procedures were administered according to the Directive 2010/63/EU of the European Parliament and the German animal protection code. Permission was approved by the Regierungspräsidium Karlsruhe, Germany (G122/12).

### NRVCM Isolation and Treatments

NRVCMs were isolated and prepared as published previously (Rangrez et al. 2013). Briefly, left ventricles from 1-2 days old Wistar rats (Charles River) were harvested and cut in pH 7.4 buffer containing 120 mmol/liter NaCl, 20 mmol/liter Hepes, 8 mmol/liter NaH₂PO₄, 6 mmol/liter glucose, 5 mmol/liter KCl, and 0.8 mmol/liter MgSO₄. The separation of single cardiomyocyte from cut tissue mass, was achieved by enzymatic digestion with 0.6 mg/ml pancreatin (Sigma) at 37° C. and 0.5 mg/ml collagenase type 11 (Worthington). Cell suspension was filtered by a cell strainer. Newborn calf serum was added to stop enzymatic digestion. In order to separate cardiomyocytes from fibroblasts, a Percoll gradient (GE Healthcare) centrifugation step was performed. Afterwards, cardiomyocytes were cultured in DMEM medium containing 10% fetal calf serum (FCS), 2 mM penicillin/streptomycin, and L-glutamine (PAA Laboratories).

NRVCMs were infected with 50 MOI (multiplicity of infection) adenovirus per cell in serum-free DMEM the next day after seeding. After 24 hours, the mediums were changed. ET-1 was applied to serum-free medium to a final concentration of 200 μmol/l for 24 hours.

L-α-Hydroxyglutaric acid disodium salt (2-HG, Sigma-Aldrich) was applied to serum-free medium to a concentration of 2 mM. Cells were harvested 2 days after treatment.

In additional experiments, NRVCMs were transduced with AAV6 vectors, either overexpressing L2HGDH or luciferase (Luc) as control at the M01=10⁵ vp/cell. Three days after transduction L-2HG (Sigma-Aldrich) was applied to cell culture medium to a final concentration of 2 mM. Cells were harvested 2 days after treatment.

### Measurement of Mitochondrial ROS Production

MitoSOX was applied to culture medium to a final concentration of 0.5 μM and cells were incubated for 15 minutes at 37° C. Afterwards, cells were washed with PBS and fixed with PFA. The fluorescence (red) was detected (Ex: 400 nm, Em at 590 nm) using confocal microscopy (Zeiss LSM 800). Mean fluorescence intensity (MFI) was calculated from the total fluorescence intensity detected from viable cells and divided by the number of cells using the software generated by Image J software (NIH, Bethesda, Md., USA).

NRVCM Staining with Mitotracker

Mitotracker Orange (Thermo Fischer Scientific) was added to cell culture medium to a concentration of 25 nmol/L. Afterwards, cells were incubated at 37° C. for 30 min, washed with PBS and fixed with 4% PFA for 5 min.

### Determination of Mitochondrial Membrane Potential

NRVCMs were maintained in μ-Slide 8-wells, (Ibidi), suitable for live cell imaging. Cells were incubated with tetramethylrhodamine (TMRE) at 37° C. to a concentration of 50 nmol/L for 30 min. Afterwards, cells were washed with warm PBS and media was replaced with FluoroBrite DMEM (Thermo Fischer Scientific). Cells were imaged within 30 min (Ex: 549; Em: 574 nm).

Similarly, JC-1 was applied to cell culture medium to a concentration of 1 μmol/L and cells were incubated at 37° C. for 30 min. After washing with warm PBS, cells were imaged in FluoroBrite DMEM (J-monomers Ex:485; Em:535/J-aggregates Ex: 530; Em:590) using a confocal microscope (LSM800, Zeiss). Mean fluorescence intensity was analysed using ImageJ.

### In Vitro Cell Surface Area Measurement

Cardiomyocytes were fixed with 4% PFA and blocked in 2.5% BSA in PBS including 0.1% Triton. Next NRVCMs were incubated with monoclonal mouse anti-α-actinin (1:200 in 2.5% BSA in PBS; Sigma) antibody for 1 hour at room temperature. After washing with PBS, secondary antibody conjugated to Alexa Fluor-546 (Thermo Fisher Scientific) together with nuclear stain DAPI (Sigma-Aldrich) at a dilution of 1:200 or 1:1000 respectively in 2.5% BSA in PBS were applied and incubated for 1 h. Afterwards, coverslips were mounted using FluorPreserve reagent (Merck Millipore). Florescence graphs were collected with Keyence fluorescence microscope BZ-9000 at ×10 magnification (objective: CFI Plan Apochromat λ×10; Nikon) and BZ-II viewer software (Keyence, version 2.1). 10 pictures were taken from each coverslip. BZ-II Analyzer (version 2.1) were used to process and analyze the graphs. HybridCellCount module and fluorescence intensity singe-extraction mode were applied for cell size measurement.

### Assessment of Hydroxymethylation Status of Cytosine

Epigenase 5mC-Hydroxylase TET Activity/Inhibition Assay Kit was employed to analyse Tet2 activity in nuclear extracts isolated from NRVCMs after 2-HG treatment. Cell fractionation was performed according to standard REAP protocol (Suzuki et al. 2010). The measurement of hydroxymethylcytosine (hmC) amount was performed according to manufacturer's instructions. 50 μg of nuclear protein was used per sample.

### Statistical Analysis

All metabolite profiling data and mRNA data were log 10-transformed before further analysis to achieve an approximate normal distribution. Missing values were not imputed for univariate analysis. Univariate analysis was performed by ANOVA (analysis of variance) using R with package nlme and the following linear model with metabolite (or gene)˜treatment+timepoint+treatment:timepoint+body weight as fixed effects. ANOVA models were read out concerning t-statistics results comprising estimates, t-values, and p-values. Significance level was set to an alpha-error of 5%. The multiple test problem was addressed by calculating the false discovery rate (FDR) using the Benjamini & Hochberg method (Benjamini and Hochberg 1995).

For the in vivo gene transfer study, the statistical analysis was performed in Sigma Plot 5 software using a one-way ANOVA and Student-Newman-Keuls' post hoc analysis (comparison between more than two groups) or unpaired t-test with Welch's correction (between two groups). For in vitro study, two-way ANOVA was performed including a Turkey's post hoc analysis when significant interaction was noted. P-values less than 0.05 were considered significant.

### EXAMPLE 2: RESULTS

### Reduced L2HDGH Expression in Various Cardiac Hypertrophy and Heart Failure Mice Models

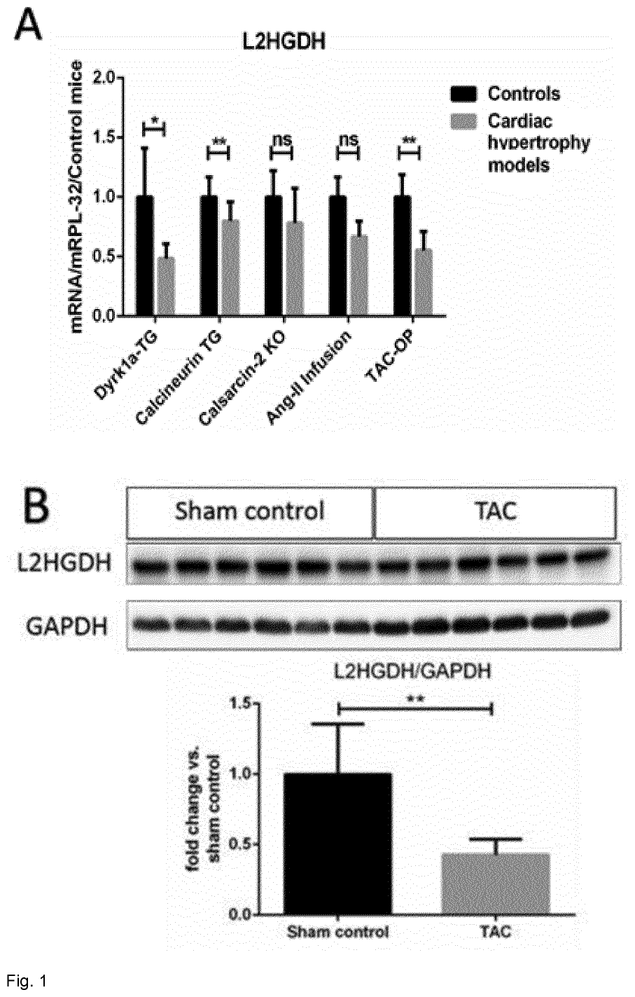

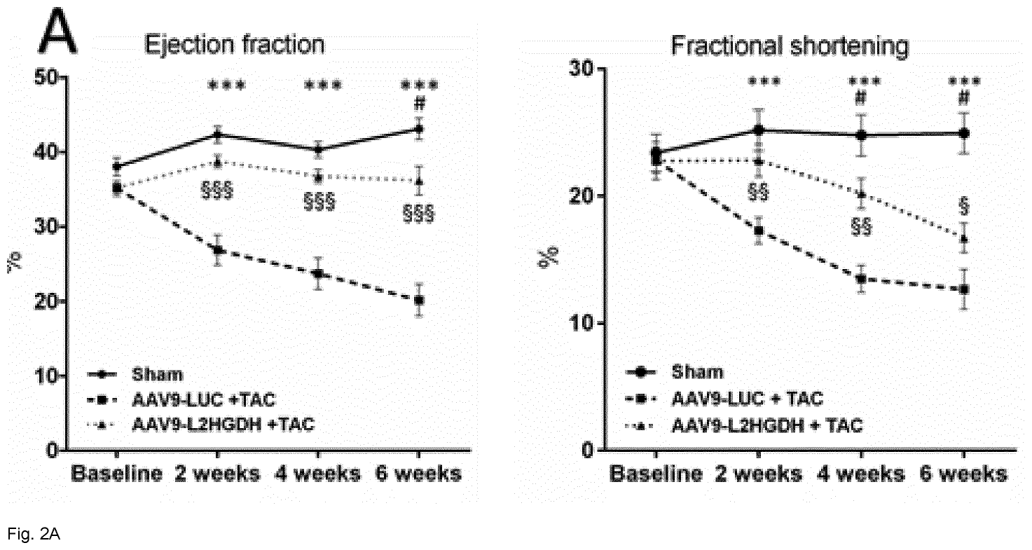

We first analysed the expression pattern of L2HDGH in various models of cardiac hypertrophy. Gene expression and protein analysis allowed us to confirm a stable L2HGDH downregulation in the analysed models as compared to controls. Notably, Dyrk1a TG mice model exhibited a 52% decrease in L2HGDH mRNA level, whereas Ang-II infusion mice had 35% down-regulation in L2HGDH mRNA level (FIG. 1A). In addition, we could find a significant decrease in L2GHDH mRNA and protein levels were found in TAC operated cardiac hypertrophy mice versus sham control (FIGS. 1A, 1B and 1C)

L2HGDH Over-Expression Alleviate Contractile Dysfunction after TAC

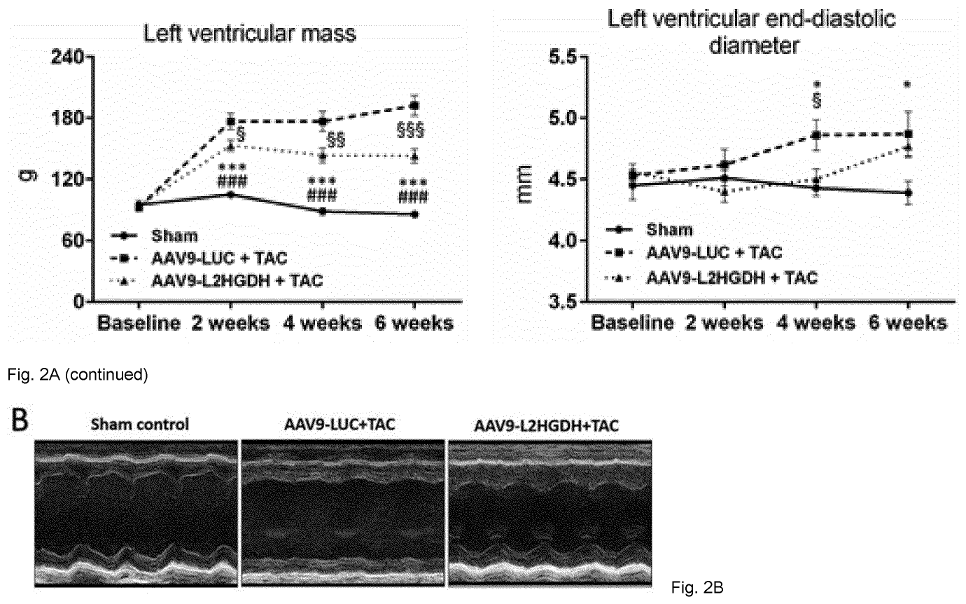

To analyse the effect of L2HGDH on heart function in a pressure-overload model of heart hypertrophy and failure in mice, the vector was injected 2 weeks prior to TAC. Follow-up echocardiography revealed significant improvement in systolic function (EF and FS) and myocardial hypertrophy (LV Mass) in the AAV9-L2HGDH group compared to AAV9-LUC group (FIG. 2). In contrast to mice receiving gene therapy, AAV9-LUC treated animals presented with marked myocardial dilatation proven by increased LVEDD at 4 and 6 weeks post-TAC, as depicted in FIG. 2A. FIG. 2B shows exemplary echocardiographic tracings.

L2HGDH Over-Expression Attenuates Pathological Remodeling after TAC

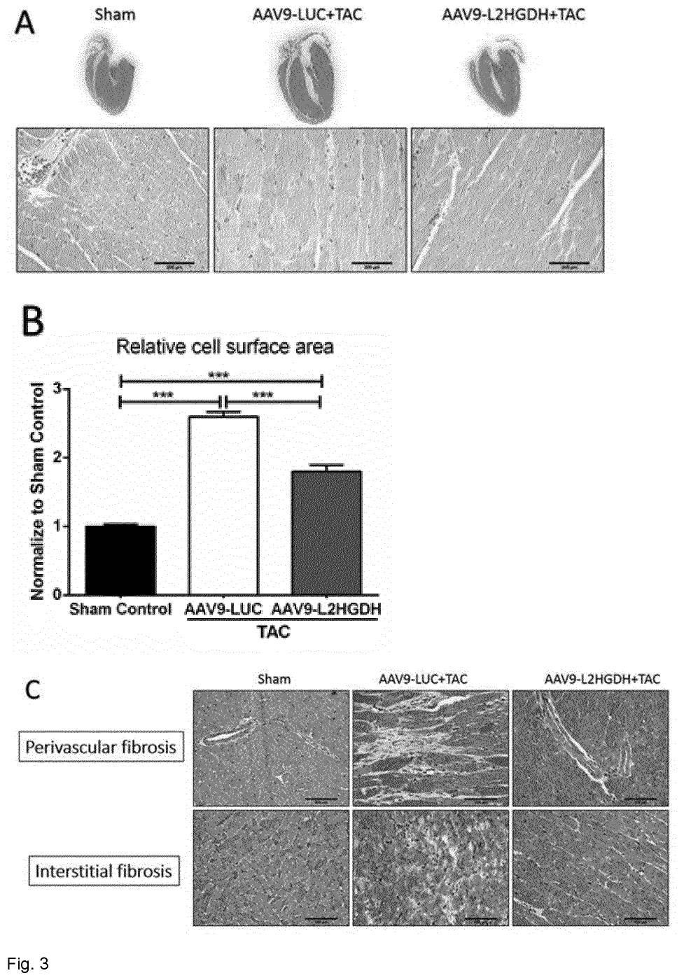

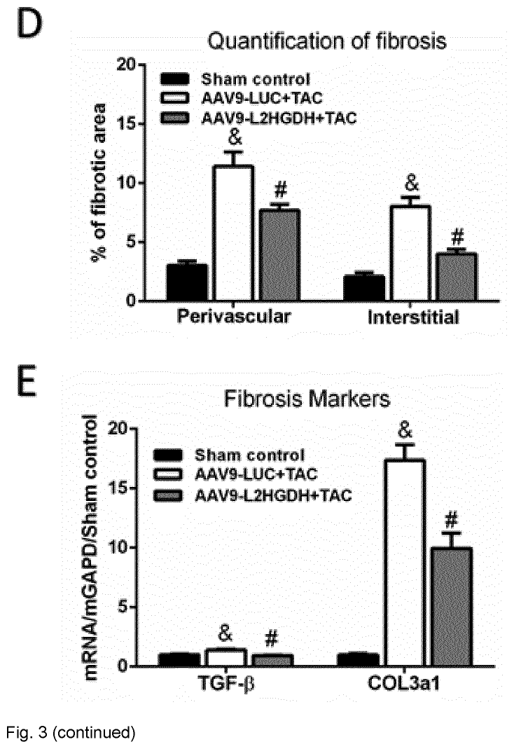

Quantification of cardiomyocyte surface area in hematoxylin/eosin (HE) staining of cardiac sections of mice subjected to TAC revealed a significant reduction in AAV9-L2HGDH treated mice compared to AAV-LUC controls (p<0.001) suggesting an attenuating effect on cardiac hypertrophy (FIGS. 3A and 3B), as observed in echocardiography.

In addition to myocyte hypertrophy, pressure overload induces intense remodeling, characterized by extracellular matrix deposition. Therefore, to assess whether L2HGDH overexpression has an effect in this context, we performed Masson's trichrome staining. As shown in FIGS. 3C and 3D, intense fibrosis was observed in hearts isolated from AAV9-LUC control mice, while AAV9-mediated L2HGDH over-expression prior to TAC significantly decreased the area of collagen deposition, both in perivascular (p<0.05) and interstitial areas (p<0.05). Additionally, fibrosis markers were analyzed on mRNA level in heart tissue. As expected, TAC resulted in a significant up-regulation of mRNA levels of transforming growth factor beta (TGF-beta) and collagen type III alpha 1 chain (COL3a1). On the other hand, when mice were injected with AAV9-L2HGDH prior to TAC, a significant decrease of TGF-beta and COL3a1 on mRNA level was detected (FIG. 3E) as compared to control AAV9-LUC injected mice.

### L2HGDH Overexpression Decreases the Mitochondrial Reactive Oxygen Species Production

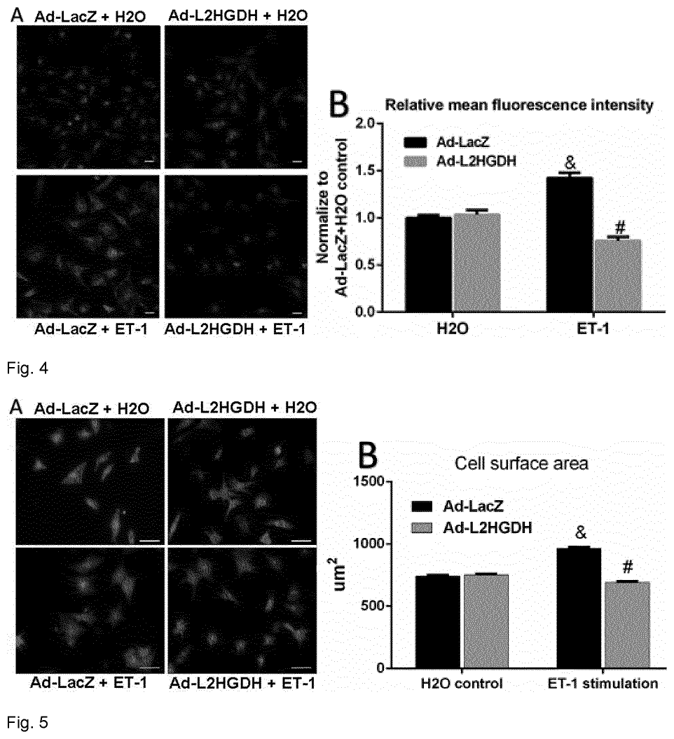

To uncover the molecular and functional mechanisms controlling L2HGDH-mediated protection, we performed in vitro experiments using primary neonatal rat ventricular cardiomyocytes (NRVCMs). As expected, ET-1 stimulation caused a significant increase in MitoSOX fluorescence level, corresponding to increased ROS production as compared to control. However, L2HGDH overexpression could normalize the generation of ROS, as pictured in FIG. 4.

### L2HGDH Overexpression Decreases ET-1 Induced Cardiomyocyte Hypertrophy In Vitro

To confirm the effect of L2HGDH of cardiomyocyte hypertrophy in vitro, we analysed NRVCMs size following transduction and ET-1 stimulation. Indeed, cardiomyocytes treated with L2HGDH-overexpressing vector presented with marked reduction in hypertrophic growth, as compared to controls (FIG. 5).

2-HG Acts as a Pro-Hypertrophic Stimulus In Vitro and Increases the Amount of 5-Hydroxymethylcytosine (5-hmC)

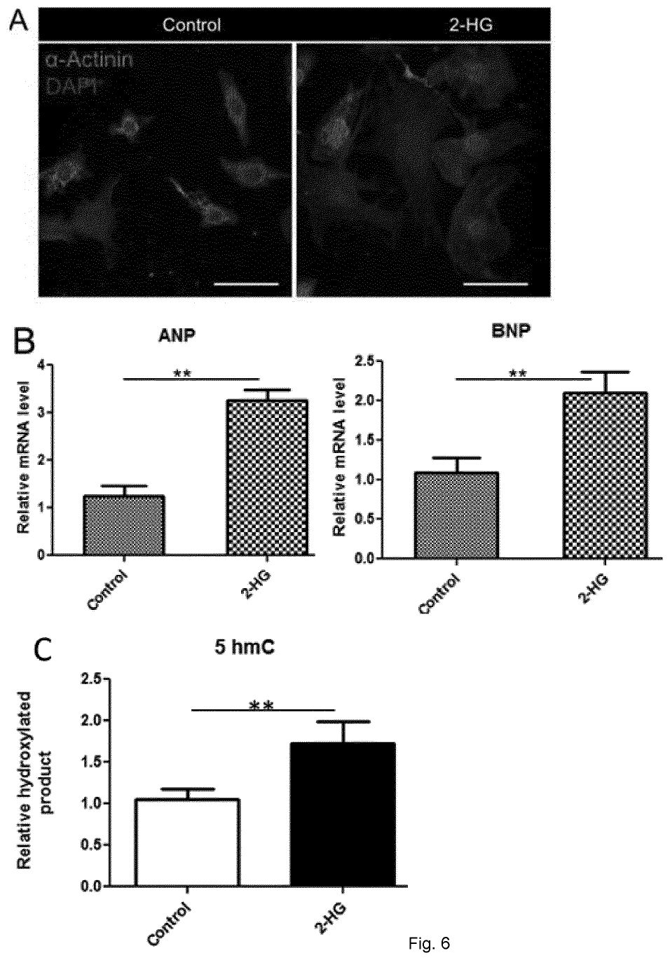

To determine whether the aforementioned results are a direct effect of increased 2-HG production due to L2HGDH downregulation in hypertrophic conditions, we applied 2-HG directly to NRVCMs and analysed the effect on cell size. As shown in FIG. 6A, 2-HG addition to cell culture medium leg to a dramatic increase in cell size as compared to non-treated cells. Moreover, we could observe a marked increase in fetal gene program, represented by ANP and BNP (FIG. 6B). Finally, it is well known that pathological cardiac hypertrophy is characterized by epigenetic alterations such as a shift towards a neonatal 5-hydroxymethylcytosine (5-hmC) distribution pattern. 2-HG treatment resulted in increased epigenetic 5-hmC deposition (FIG. 6C), further supporting a causal role of 2-HG in pathological hypertrophy (Greco et al. 2016).

AAV6-Mediated L2HGDH Overexpression in Cardiomyocytes is Translocated into the Mitochondria

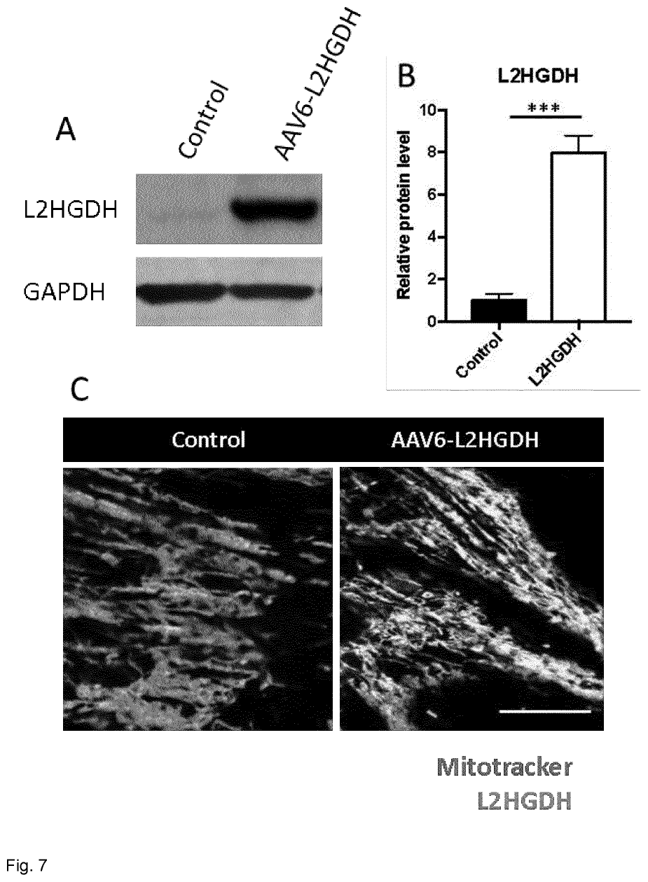

First, we aimed to determine the degree of L2HGDH overexpression in NRVCMs following AAV6 transduction. As shown in FIG. 7A, B, Western blot experiments prove an 8-fold increased L2HGDH protein level in lysates of primary cells transduced with AAV6-L2HGDH. Taking into account that L2HGDH is primarily located within the inner membrane of the mitochondria (Rzem et al. Proc Natl Acad Sci USA; 101:16849-54), we next performed colocalization studies of L2HGDH and Mitotracker. As proven in FIG. 7C, confocal images could clearly demonstrate that the overexpressed L2HGDH is located in the mitochondria after NRVCM transduction.

### L2HGDH Overexpression Ameliorates 2HG Induced Cardiomyocyte Hypertrophy In Vitro

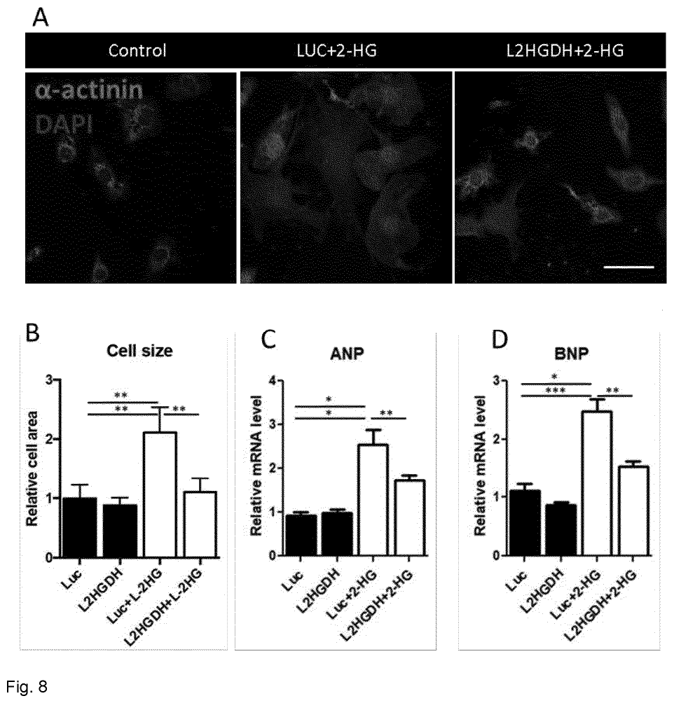

We next wanted to establish the role of 2HG in cardiac hypertrophy in vitro. 2HG application caused a marked induction of hypertrophy in NRVCMs, as proven by increased cell size and mRNA levels of well-established pro-hypertrophic markers (FIG. 8). On the other hand, transduction with L2HGDH-overexpressing AAV6 prior to metabolite addition to culture medium led to a marked decreased hypertrophic growth and reduced mRNA levels of ANP and BNP.

### L2HGDH Overexpression Blunts Mitochondrial Dysfunction Caused by 2HG

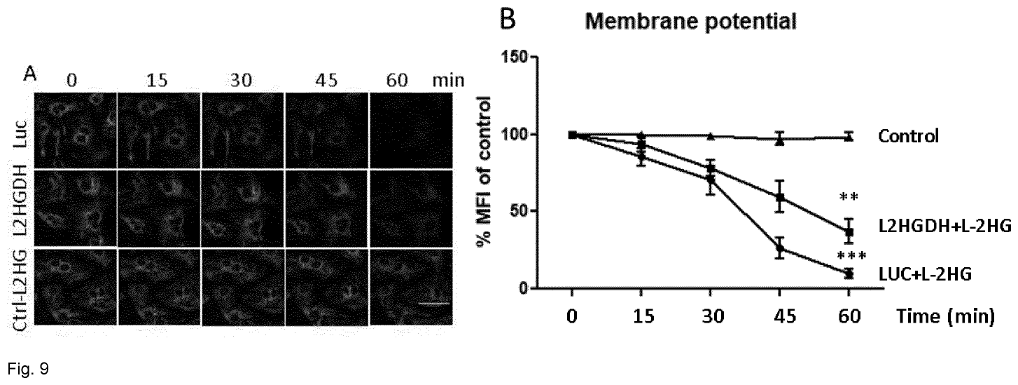

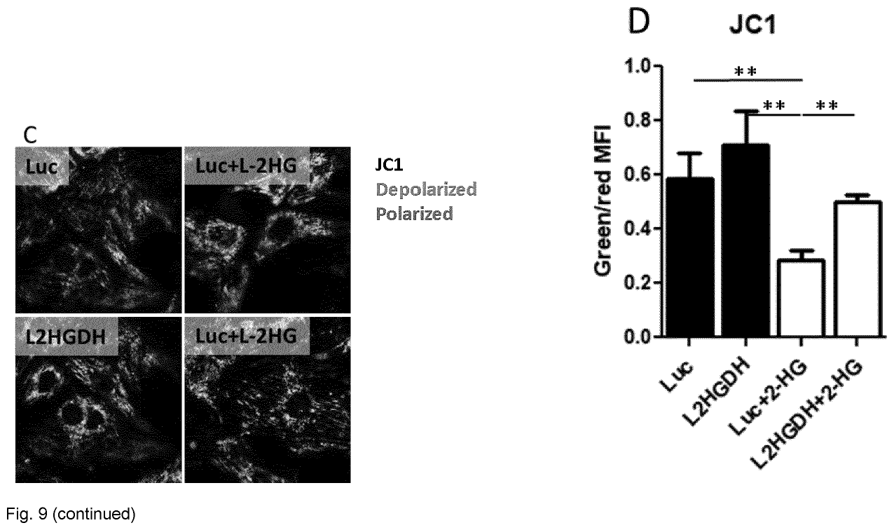

Given that mitochondrial dysfunction is one of the main drivers of pathological hypertrophy (Zhou et al. J Clin Invest 128:3716-26), we further evaluated mitochondrial activity in NRVCMs following L-2HG treatment. As shown is FIG. 9 A, B, 2HG caused a time-dependent decrease in TMRE fluorescence, proving a likewise reduction in mitochondrial membrane potential. In stark contrast, L2HGDH-overexpressing NRVCMs presented with increased mitochondrial activity as compared to Luc transduced cells. To further corroborate these findings, we next performed JC-1 stainings and measured the ratio of green (depolarized mitochondria) to red (polarized mitochondria) mean fluorescence intensity. Our results demonstrate that 2HG treatment resulted in mitochondrial depolarization, while L2HGDH overexpression preserved its function (FIG. 9 C,D).

### L-2HG Induces Fibroblast Proliferation and Collagen Synthesis

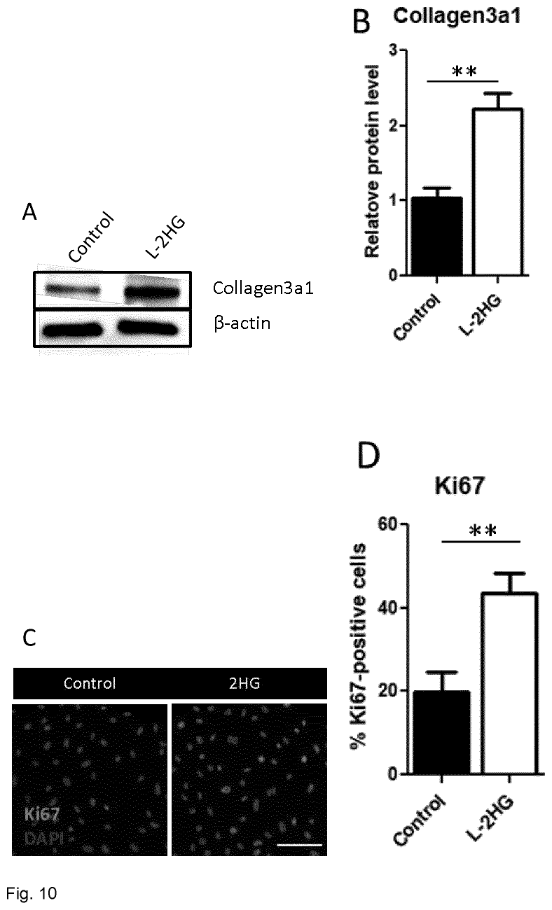

We further addressed whether L-2HG affects other resident cells in the myocardium. Fibroblasts respond to stress stimuli by increasing their rate of proliferation and synthesis of extracellular matrix, further leading to development of pathological fibrosis (Moore-Morris et al. J Mol Med 93:823-30). Our data underlines that L-2HG application to primary rat neonatal fibroblasts induces a significant increased collagen3a1 expression (FIG. 10A, B). Moreover, L-2HG treatment intensified fibroblasts proliferation, as proven by a dramatic increased Ki67-positive nuclei following treatment (FIG. 10C,D).

### LITERATURE

1. Ponikowski, P., et al., 2016 ESC Guidelines for the diagnosis and treatment of acute and chronic heart failure: The Task Force for the diagnosis and treatment of acute and chronic heart failure of the European Society of Cardiology (ESC). Developed with the special contribution of the Heart Failure Association (HFA) of the ESC. Eur J Heart Fail, 2016. 18(8): p. 891-975.
2. Goldberg L R. In the clinic. Heart failure. Ann Intern Med. 2010 Jun. 1; 152(11): ITC 61-15
3. Müller, O J., et al., Comprehensive plasma and tissue profiling reveals systemic metabolic alterations in cardiac hypertrophy and failure. Cardiovasc Res. 2018 Nov. 9. doi: 10.1093/cvr/cvy274. [Epub ahead of print]
4. Kranendijk, M., et al., Progress in understanding 2-hydroxyglutaric acidurias.

Journal of inherited metabolic disease, 2012. 35(4): p. 571-587.
5. Moroni, I., et al., L-2-hydroxyglutaric aciduria and brain malignant tumors A predisposing condition? Neurology, 2004. 62(10): p. 1882-1884.
6. Shim, E.-H., et al., L-2-Hydroxyglutarate: an epigenetic modifier and putative oncometabolite in renal cancer. Cancer discovery, 2014. 4(11): p. 1290-1298.
7. Latini, A., et al., Mitochondrial energy metabolism is markedly impaired by D-2-hydroxyglutaric acid in rat tissues. Molecular genetics and metabolism, 2005. 86(1): p. 188-199.
8. Fu, X., et al., 2-Hydroxyglutarate inhibits ATP synthase and mTOR signaling. Cell metabolism, 2015. 22(3): p. 508-515.
9. Intlekofer, A. M., et al., Hypoxia induces production of L-2-hydroxyglutarate. Cell metabolism, 2015. 22(2): p. 304-311.
10. Oldham W M, Clish C B, Yang Y, Loscalzo J. Hypoxia-Mediated Increases in L-2-hydroxyglutarate Coordinate the Metabolic Response to Reductive Stress. Cell Metab. 2015, 22(2):291-303.

11. Bensaad, K. and A. L. Harris, Hypoxia and metabolism in cancer, in Tumor Microenvironment and Cellular Stress. 2014, Springer. p. 1-39.
12. Müller, O. J., et al., Targeting the heart with gene therapy-optimized gene delivery methods. Cardiovasc Res, 2007. 73(3): p. 453-62.

13. Werfel, S., et al., Rapid and highly efficient inducible cardiac gene knockout in adult mice using AAV-mediated expression of Cre recombinase. Cardiovasc Res, 2014. 104(1): p. 15-23.
14. Heckmann M B, Bauer R, Jungmann A, Winter L, Rapti K, Strucksberg K-H, Clemen C S, Li Z, Schröder R, Katus H A, Müller OJ. AAV9-mediated gene transfer of desmin ameliorates cardiomyopathy in desmin-deficient mice. Gene Ther 2016; 23:673-679.
15. Fischer, A. H., et al., Hematoxylin and eosin staining of tissue and cell sections. CSH Protoc, 2008. 2008: p. pdb prot4986.
16. Bickelhaupt, S., et al., Effects of CTGF Blockade on Attenuation and Reversal of Radiation-Induced Pulmonary Fibrosis. J Natl Cancer Inst, 2017. 109(8).
17. deAlmeida, A. C., et al., Transverse aortic constriction in mice. J Vis Exp, 2010(38).
18. Rangrez, A. Y., et al., Dysbindin is a potent inducer of RhoA-SRF-mediated cardiomyocyte hypertrophy. J Cell Biol, 2013. 203(4): p. 643-56.
19. Suzuki, K., et al., REAP: A two minute cell fractionation method. BMC Res Notes, 2010. 3: p. 294.
20. Benjamini Y, Hochberg Y. Controlling the False Discovery Rate: A Practical and Powerful Approach to Multiple Testing. Source J R Stat Soc Ser B 1995; 57:289-300.
21. Greco C M, Kunderfranco P, Rubino M, Larcher V, Carullo P, Anselmo A, Kurz K, Carell T, Angius A, Latronico M V, Papait R, Condorelli G. DNA hydroxymethylation controls cardiomyocyte gene expression in development and hypertrophy. Nat Commun. 2016; 7:12418.
22. Rzem R, Veiga-da-Cunha M, Noel G, et al.: A gene encoding a putative FAD-dependent L-2-hydroxyglutarate dehydrogenase is mutated in L-2-hydroxyglutaric aciduria. P Natl Acad Sci USA 2004; 101:16849-54.
23. Zhou B, Tian R: Mitochondrial dysfunction in pathophysiology of heart failure. J Clin Invest 2018; 128:3716-26.
24. Moore-Morris T, Guimaraes-Camboa N, Yutzey K E, Puceat M, Evans S M: Cardiac fibroblasts: from development to heart failure. J Mol Med 2015; 93:823-30.
25. Ban et al.: A Novel Malate Dehydrogenase 2 Inhibitor Suppresses Hypoxia-InducibleFactor-1 by Regulating Mitochondrial Respiration, PLoS One, doi:10.1371/journal.pone.0162568 (2016)
26. Shelar et al.: Biochemical and Epigenetic Insights into L-2-Hydroxyglutarate, a Potential Therapeutic Target in Renal Cancer, Clin Cancer Res 24(24):6433; doi:10.1158/1078-0432. CCR-18-1727 (2018)

1-26. (canceled)

27. A method for treating, preventing, and/or preventing progression of cardiac remodeling, in particular cardiomyopathy and/or heart failure in a subject, comprising:
(a) contacting the subject with a compound that decreases the concentration of 2-hydroxy-glutarate (2HG); and
(b) thereby treating, preventing, and/or preventing progression of cardiac remodeling.

28. The method of claim 27, wherein the 2HG is L-2-hydroxy-glutarate (L2HG).

29. The method of claim 27, wherein the concentration is blood concentration and/or intracellular concentration in cells of the circulatory system.

30. The method of claim 27, wherein the concentration is in cells of the heart, preferably in cardiomyocytes.

31. The method of claim 27, wherein the compound is an enzyme catalyzing degradation of 2HG, preferably an 2HG-Dehydrogenase (2HGDH).

32. The method of claim 31, wherein the compound is an enzyme catalyzing degradation of L2HG, preferably an L2HG-Dehydrogenase (L2HGDH).

33. The method of claim 31, wherein the compound is an L2HGDH enzyme EC 1.1.99.2.

34. The method of claim 27, wherein the compound is a polynucleotide comprising an expressible sequence encoding a 2HGDH, preferably an L2HGDH.

35. The method of claim 27, wherein the compound is a polynucleotide comprising an expressible sequence encoding a 2HGDH, preferably an L2HGDH, comprised in a viral particle, preferably an adeno-associated virus (AAV) particle, more preferably in an AAV9 particle.

36. The method of claim 27, wherein the polynucleotide comprises
(a) the nucleic acid sequence of SEQ ID NO:1 and/or 3 or a nucleic acid sequence at least 70% identical to at least one of SEQ ID NO:1 and/or 3; and/or
(b) a nucleic acid sequence encoding a polypeptide comprising the amino acid sequence of SEQ ID NO: 2 and/or 4; or encoding a polypeptide comprising an amino acid sequence at least 70% identical to at least one of SEQ ID NO:2 and/or 4.

37. The method of claim 27, wherein the said polynucleotide comprises the nucleic acid sequence of SEQ ID NO:5 or a nucleic acid sequence at least 70% identical thereto; preferably comprises the nucleic acid sequence of SEQ ID NO:5, more preferably consists of the nucleic acid sequence of SEQ ID NO:5.

38. The method of claim 27, wherein the cardiac remodeling is caused by (i) arterial hypertension; (ii) congenital, age-related degenerative, or infection-related semilunar valve stenosis, in particular aortic valve stenosis; (iii) cardiomyopathy, in particular dilated cardiomyopathy, hypertrophic cardiomyopathy, arrhythmogenic right ventricular cardiomyopathy, left ventricular noncompaction, or restrictive cardiomyopathy; (iv) coronary heart disease; or (v) myocarditis.

39. The method of claim 27, wherein the subject is a mammal, preferably a human.

40. A viral particle comprising a polynucleotide comprising an expressible sequence encoding a 2HGDH, preferably an L2HGDH.

41. The viral particle of claim 40, wherein the viral particle is a virus-like particle.

42. The viral particle of claim 40, wherein the viral particle is an adeno-associated virus (AAV) particle, more preferably is an AAV9 particle.

43. The viral particle of claim 40, comprised in a pharmaceutical composition.

44. The viral particle according to claim 43, wherein the pharmaceutical composition is for use in treating, preventing, and/or preventing progression of cardiac remodeling, in particular cardiomyopathy and/or heart failure.

45. A kit comprising:
(a) a polynucleotide comprising an expressible sequence encoding a 2HGDH, preferably an L2HGDH, and at least one viral packaging signal, and
(b) a corresponding packaging helper polynucleotide and/or a corresponding packaging cell line.
